(19) United States
(12) Patent Application Publication
(10) Pub. No.: US 2005/0276825 A1
(43) Pub. Date: Dec. 15, 2005

(54) Single oil impregnated post bath towels

(76) Inventor: Kenneth Davidson, Dayton, OH (US)

Correspondence Address: William M. Selenke, 28 DeWitt St., Cincinnati, OH, 45218, US

(21) Appl. No.: 10/429,778

(22) Filed: May 5, 2003

Related U.S. Application Data
(60) Provisional application No. 60/378,461, filed on May 7, 2002.

(51) Int. Cl. A61K 7/00

(52) U.S. Cl. 424/401

(57) ABSTRACT

The invention is an oil impregnated towel of either woven or non woven fibers that contain a single emolument oil chosen from the group that includes olive oil, mineral oil, cocoa butter, or vitamin E oil. The towels arc dispensed one at a time from containers that isolate the towel from the ambient environment. The towels may be dispensed in single sealed packets, or the towels may be dispensed from an oil resistant boxes wherein the single oil impregnated towels are manufactured to be dispensed one at a time from an ordered array of folded towels.

### FIELD OF INVENTION

The present invention is concerned with commercially prepared wipes to provide a smooth oil emollient to the skin after a regular water bath or shower. Of particular concern are the natural emollients olive oil (sweet oil), cocoa butter, Vitamin E in oil, and mineral oil. It is to be noted that the oils are placed on a longer towel-like construct so that the small of the back as well as the more readily accessible sites can receive the post bath emollient.

### BACKGROUND OF THE INVENTION

The process of bathing with soap typically removes the natural oils from the body. Of course, the natural oils are often fill of bacteria and other odor causing agents. After a soapy bath and towel drying the body is left with lesser amounts of natural oils to maintain moisture in and elasticity of the human skin. There are a number of solutions to this problem. In some instances oils as are added to the bath water and the residual oils will remain on the skin. In nursing homes the client receives a rubdown with body lotions after a bath. There are a number of patents for various towels and pads to place emollients of oil on the human skin. For example, Davidson application Ser. No. 10/121,755 describes the use of olive oil on pads and on swabs to be used to add oils to the skin. This application is incorporated by reference. The present invention is concerned not only with olive oil but with cocoa butter, mineral oil, and vitamin E oil. More specifically, agents as individual oils are impregnated into larger disposable towels capable of being pulled across the small and other parts of the human back. This larger towel may be used to add oil to other parts of the body. Moreover, the oil impregnated towels for post bath application may be smaller.

Various fabrics both woven and non woven are widely known in commercial channels and described in the patent literature. For purposes of the present invention, commercially available fabrics that are compatible with the oils of the present invention may be used. Likewise, the impregnation of oils into these fabrics are standard procedures well-known to the art. Likewise, the individual towels may be packed in individual containers, as well as dispensed from containers that store the folded towels in the manner that as is commonly used for individual nasal tissues.

The present invention is concerned with towels the are large enough so that the individual may apply the oils namely olive oil, cocoa butter, pharmaceutical grade mineral oil, or vitamin E oil as individual oils to the back and other surfaces of the individual. Clearly such towels would be relatively long compared to a relatively narrow width.

### BRIEF DESCRIPTION OF FIGURES

FIG. 1 shows the oil impregnated towel in a single package container.

FIG. 2 and FIG. 3 shows alternative views of a container/dispenser for single towels from a stacked ordered array of towels.

### DETAILED DESCRIPTION OF THE FIGURES

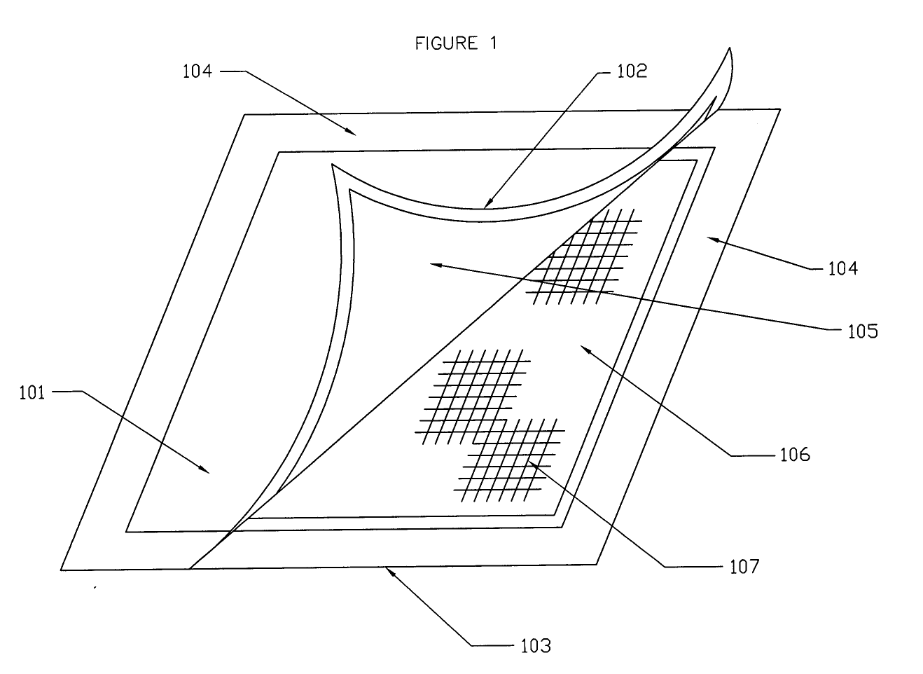

FIG. 1 shows a sealed packet 101 as the single package container to isolate the oil impregnated towel from the ambient environment. Packet 101 has a front side 102 and a back side 103. During manufacture front side 102 and back side 103 are sealed via seal 104. In the compartment 105 is placed a pad of absorbent material 106 such as cotton gauze or other woven or non woven fabric. Fabric towel 206 is saturated with olive oil, mineral oil, cocoa butter, or vitamin E oil 107. This sealed packet 101 is shown partially opened to access the gauze 106 saturated with appropriate single oil 107.

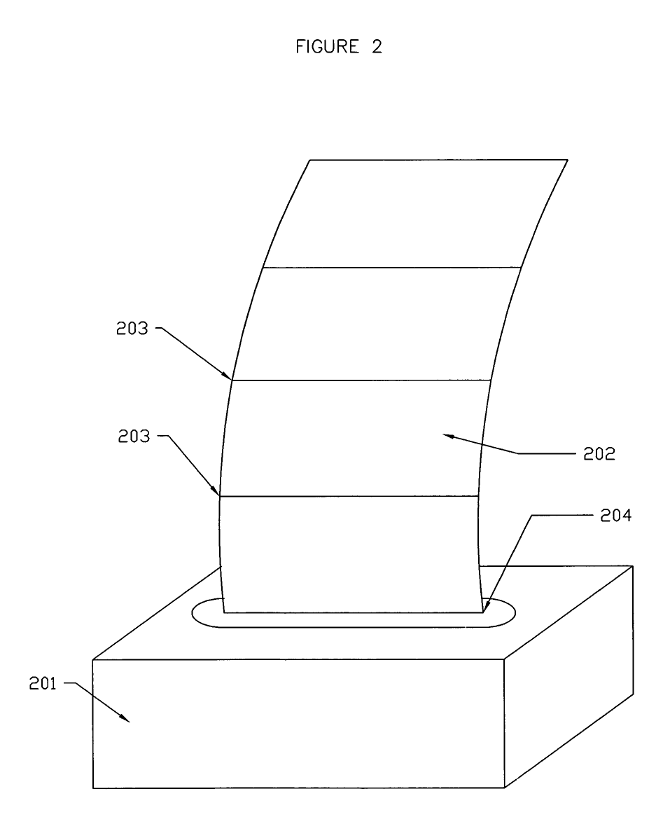

FIG. 2 illustrates a oil proof dispenser box 201 as a container to isolate the towels from the ambient environment and to dispense oil impregnated towels one at a time. Box 201 is a generally bricked shaped box, but other shapes are possible. Internal to box 201 are the oil impregnated towels arranged so that the oil impregnated towels 202 are dispensed one at a time through opening 204. Creases 203 show that in the present embodiment the oil impregnated towels are folded and are unfolded as they are removed from box 201. Other means of arranging the towels 202 are known to the art.

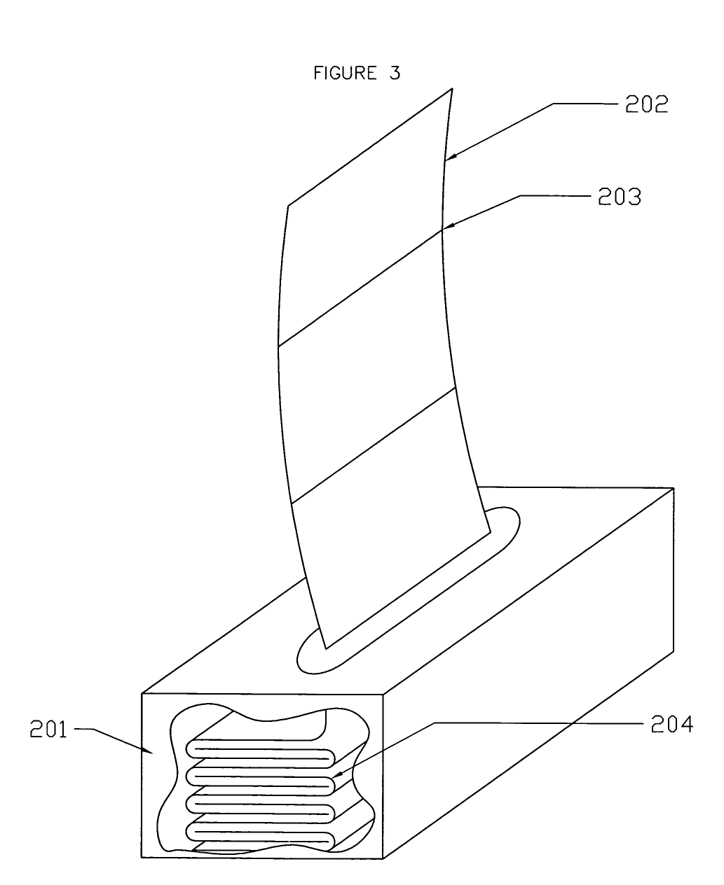

FIG. 3 show an edge perspective of the oil proof dispenser box 201. The oil impregnated towels are shown as dispensed towels 202 with folds 203. 204 is an outline of the ordered array of folded towels folded to be removed one at a time.

### SUMMER OF INVENTION

The invention is an oil impregnated towel of either woven or non woven fibers that contain a single emolument oil chosen from the group that includes olive oil, mineral oil, cocoa butter, or vitamin E oil. The towels are dispensed one at a time from containers that isolate the towel from the ambient environment. The towels may be dispensed in single sealed packets, or the towels may be dispensed from an oil resistant boxes wherein the single oil impregnated towels are manufactured to be dispensed one at a time from an ordered array of folded towels.

1) An oil impregnated towel to be used after a bath comprising:
a fabric towel of either woven or non woven fibers containing a single emolument oil chosen from the group that includes olive oil, mineral oil, cocoa butter, or vitamin E oil;
said towels are dispensed one at a time from containers that isolate the towel from the ambient environment.

2) An oil impregnated towel to be used after a bath as in claim 1 wherein said single emolument oil is olive oil (sweet oil).

3) An oil impregnated towel to be used after a bath as in claim 1 wherein said single emolument oil is cocoa butter.

4) An oil impregnated towel to be used after a bath as in claim 1 wherein said single emolument oil is pharmaceutical grade mineral oil.

5) An oil impregnated towel to be used after a bath as in claim 1 wherein said single emolument oil is Vitamin E oil.

6) An oil impregnated towel to be used after a bath as in claim 1 wherein said containers dispense towels in single sealed packets.

7) An oil impregnated towel to be used after a bath as in claim 1 wherein said towels are dispensed from an oil resistant boxes and said towels are manufactured to be dispensed one at a time from an ordered array of folded towels.
